(19) United States
(12) Patent Application Publication
(10) Pub. No.: US 2008/0177239 A1
(43) Pub. Date: Jul. 24, 2008

(54) TROCAR CANNULA SYSTEM

(76) Inventors: Yong Li, Valencia, CA (US); Christopher L. McCollam, Irvine, CA (US)

Correspondence Address: ALCON, IP LEGAL, TB4-8, 6201 SOUTH FREEWAY, FORT WORTH, TX, 76134, US

(21) Appl. No.: 11/624,485

(22) Filed: Jan. 18, 2007

(51) Int. Cl. A61M 25/00

(52) U.S. Cl. 604/264

(57) ABSTRACT

A trocar cannula system that exhibits improved retention of the microsurgical instrument within the trocar cannula.

### FIELD OF THE INVENTION

The present invention generally pertains to microsurgical instruments. More particularly, but not by way of limitation, the present invention pertains to microsurgical instruments used in posterior segment ophthalmic surgery.

### DESCRIPTION OF THE RELATED ART

Many microsurgical procedures require precision cutting and/or removal of various body tissues. For example, vitreoretinal surgery often requires the cutting, removal, dissection, delamination, coagulation, or other manipulation of delicate tissues such as the vitreous humor, traction bands, membranes, or the retina. The vitreous humor, or vitreous, is composed of numerous microscopic fibers that are often attached to the retina. Therefore, cutting, removal, or other manipulation of the vitreous must be done with great care to avoid traction on the retina, the separation of the retina from the choroid, a retinal tear, or, in the worst case, cutting and removal of the retina itself.

Microsurgical instruments, such as vitrectomy probes, fiber optic illuminators, infusion cannulas, aspiration probes, scissors, forceps, and lasers are typically utilized during vitreoretinal surgery. These devices are generally inserted through one or more surgical incisions in the sclera near the pars plana, which are called sclerotomies. The repeated insertion and removal of these devices can allow vitreous and other fluids to escape the eye through the sclerotomies, increasing the potential for softening of the globe, bleeding, traction on the retina, or introduction of bacteria into the eye. Therefore, an infusion cannula is used to infuse an irrigating solution into the eye to maintain a suitable intraocular pressure.

Trocar cannulas and plugs are often used in connection with infusion cannulas. The trocar cannula establishes an entry through the sclera into the posterior segment of the eye. The infusion cannula is placed within the trocar cannula so as to provide irrigating solution to the eye. A trocar plug is placed within the trocar cannula to prevent leakage when the infusion cannula is removed. It is important to establish a reliable connection between the trocar cannula and the infusion cannula or trocar plug to prevent an unwanted loss of intraocular pressure during surgery. Typically, such connection is accomplished via an interference fit between the inner diameter of the trocar cannula and the outer diameter of the infusion cannula or trocar plug. However, interferences may develop inelastic deformation of, and create static friction between, the trocar cannula and the infusion cannula or trocar plug. Such interferences are also difficult to control with small gage instruments and conventional manufacturing tolerances. In addition, a conventional method of creating such an interference is by crimping the infusion cannula. However, such crimping often creates a flow restriction within the infusion cannula, which is particularly undesirable during the high vacuum settings of some surgeries. Therefore, a need remains for an improved trocar cannula system.

### SUMMARY OF THE INVENTION

In one aspect, the present invention is a trocar cannula system that generally includes a trocar cannula and a microsurgical instrument. The trocar cannula has a hub and a tubing coupled to the hub. The tubing has an internal surface with a protrusion disposed thereon. The microsurgical instrument is removably and partially disposed within the tubing. The instrument has an external surface with a well disposed thereon for mating with the protrusion. The mating of the protrusion and the well place the instrument in three point bending to secure the instrument within the trocar cannula.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is schematic, side, sectional view of the trocar cannula system according to the present invention; and

FIG. 2 is an enlarged, side, sectional view of a portion of the infusion cannula of the trocar cannula system shown in detail 2 of FIG. 1.

### DETAILED DESCRIPTION OF THE PREFERRED EMBODIMENTS

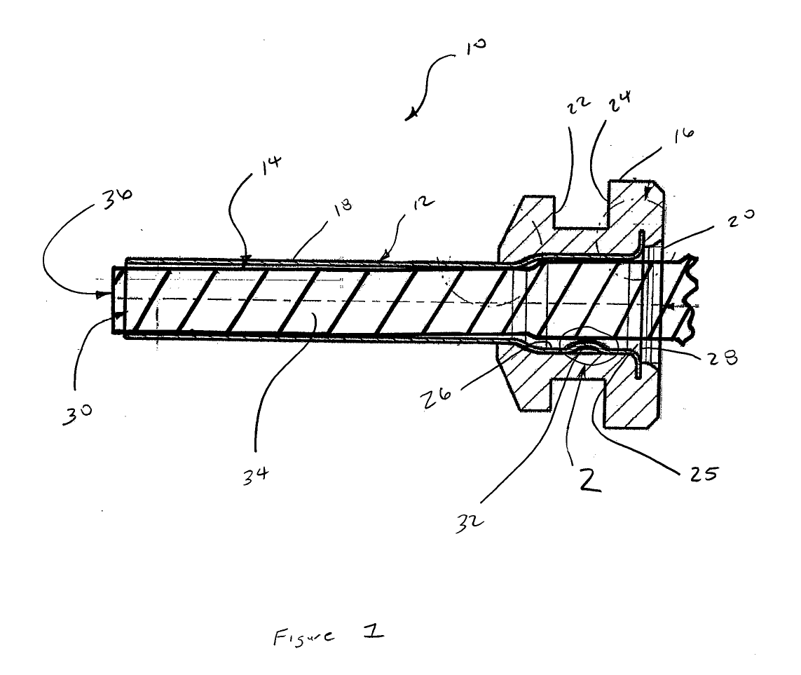

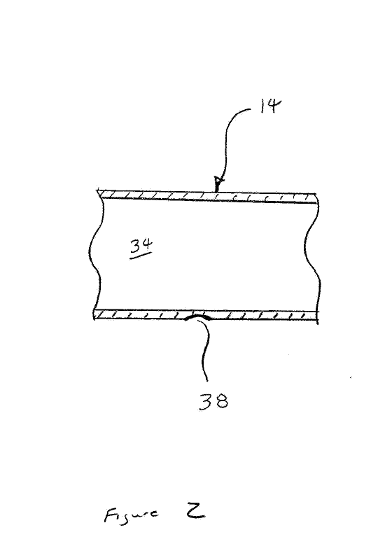

The preferred embodiments of the present invention and their advantages are best understood by referring to FIGS. 1-2 of the drawings, like numerals being used for like and corresponding parts of the various drawings.

FIG. 1 shows a trocar cannula system 10 generally including a trocar cannula 12 and an infusion cannula or trocar plug 14. For ease of description, the present invention will be described below in reference to an infusion cannula with the understanding that it is equally applicable to a trocar plug or other microsurgical instrument.

Trocar cannula 12 generally includes a proximal hub 16 and a tubing 18. Hub 16 includes an opening 20 for receiving infusion cannula 14, an annular surface 22 for contacting an interior surface of the sclera, and an annular surface 24 for contacting an exterior surface of the sclera. Tubing 18 has a hollow bore 25, an internal surface 26, an opening 28 for receiving infusion cannula 14, and an opening 30 for allowing the passage of infusion cannula 14 into the posterior segment of the eye. Tubing 18 has a first portion disposed within hub 16 having a first internal diameter and a second, distal portion with a second internal diameter smaller than its first internal diameter. Internal surface 26 has a convex protrusion 32, which is preferably disposed within hub 16. Trocar cannula 12 is preferably formed from surgical stainless steel.

Infusion cannula 14 generally includes a hollow bore 34, an opening 36 for allowing irrigating fluid to enter the posterior segment of the eye, a first portion disposed within hub 16 having a first internal diameter, and a second, distal portion with a second internal diameter smaller than its first internal diameter. As shown best in FIG. 2, infusion cannula 14 has a concave well or dimple 36 on its external surface for mating with protrusion 32 of trocar cannula 12. Infusion cannula 14 is preferably formed from surgical stainless steel.

In use, a surgeon creates a sclerotomy and inserts trocar cannula 12 therein so that the sclera is disposed between surfaces 22 and 24 of hub 16; opening 30 is disposed within the posterior segment, and preferably the vitreous chamber, of the eye; and opening 20 is disposed outside the sclera. The surgeon inserts infusion cannula 14 into tubing 18 so that opening 36 is disposed within the posterior segment, and preferably the vitreous chamber, of the eye. Protrusion 32 of trocar cannula 12 mates with well 36 of infusion cannula 14. The mating of protrusion 32 and well 36 places infusion cannula 14 in elastic, three point bending, safely securing infusion cannula 14 within trocar cannula 12 with a reasonable retention force. The mating of protrusion 32 and well 36 creates no flow restricting features within bore 34 of infusion cannula 14. The mating of protrusion 32 and well 36 can be disengaged with a reasonable amount of force to allow for the removal of infusion cannula 14. A trocar plug 14, or other microsurgical instrument, may then be inserted into the sclerotomy.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

1. A trocar cannula system, comprising:
a trocar cannula having a hub and a tubing coupled to said hub, said tubing having an internal surface with a protrusion disposed thereon;
a microsurgical instrument partially disposed within said tubing, said instrument having an external surface with a well disposed thereon for mating with said protrusion;
wherein said mating of said protrusion and said well place said instrument in three point bending to removably secure said instrument within said trocar cannula and to insure that said instrument can be removed from said trocar cannula with a predetermined amount of force.

2. The trocar cannula system of claim 1 wherein said protrusion has a convex geometry.

3. The trocar cannula system of claim 2 wherein said well has a concave geometry.

4. The trocar cannula system of claim 1 wherein said microsurgical instrument is an infusion cannula.

5. The trocar cannula system of claim 1 wherein said microsurgical instrument is a trocar plug.

6. (canceled)

7. (canceled)

8. (canceled)
